(19) United States
(12) Patent Application Publication
(10) Pub. No.: US 2007/0099156 A1
(43) Pub. Date: May 3, 2007

(54) Self-etching dental primer compositions and methods and systems utilizing such compositions

(76) Inventors: Jeff Wagner, Salt Lake City, UT (US); Dan E. Loveridge, Sandy, UT (US)

Correspondence Address: Rick D. Nydegger;WORKMAN NYDEGGER, 1000 Eagle Gate Tower, 60 East South Temple, Salt Lake City, UT, 84111, US

(21) Appl. No.: 11/261,171

(22) Filed: Oct. 28, 2005

(51) Int. Cl. A61K 6/08

(52) U.S. Cl. 433/228.1; 106/35; 523/116

(57) ABSTRACT

Two-part self-etching primer compositions can be used to etch and prime a tooth surface. The composition includes an acid component comprising a polymerizable organic acid contained in a first part, and a polymerizable component comprising at least one acrylate or methacrylate resin in a second part. A self-etching primer composition is formed upon mixing the first part with the second part. The self-etching primer composition may include a photoinitiator and/or other optional components.

### BACKGROUND OF THE INVENTION

1. The Field of the Invention

The present invention is in the field of acid etch and primer compositions, more particularly etching compositions used in etching teeth and priming compositions used in preparing or priming a surface for application of another composition (e.g., a sealant or a bonding composition).

2. The Relevant Technology

In the course of performing certain dental procedures it is often necessary to bond a dental prosthetic or other device to a surface of a tooth. In order to provide for a strong bond between the existing dental tissue and the device, a dental practitioner will first prepare the tooth surface. According to one such method, the dental practitioner first applies an acidic etching composition to exposed tooth enamel and dentin to clean and etch the tissue surface.

Bonding resin compositions are typically hydrophobic, and any water or moisture present at the bonding site must be dried in order to obtain a strong bond between the exposed enamel and dentin and the bonding resin. While it can be relatively simple to dry an enamel surface, it is much more difficult to obtain a completely dry dentin surface by using a drying agent and/or air drying techniques. Dentin is more difficult to dry and keep dry compared to enamel because dentin contains thousands of dentinal tubules which radiate outward from the pulp of the tooth and which contain pulp fluid under pressure. A cross-section of dentin contains approximately 35,000 dentin tubules per square millimeter, which can ooze fluids from the pulp after the dentin surface is cut or drilled and the smear layer has been altered or removed.

Cutting or drilling the dentin creates a “smear layer,” which is a semi-attached, weakened layer of dentin. Tests have shown that it is difficult to obtain strong bonds between resinous materials and dentin unless the smear layer has been substantially removed. This may be accomplished by the use of the etching composition.

A dental primer including a polymerizable resin may then be applied so as to displace moisture from the surface of the dentin. The hydrophobic bonding resin may then be applied to the preparation site, and a sufficiently strong bond to the existing tooth structure can be obtained.

More recent developments have attempted to reduce the number of steps and separate compositions required with the standard 3-step process described above (etch, prime, and bond).

Some systems have been developed that include application of a single etching and priming composition to both etch and prime the desired site. Such compositions may include an acid etchant component for etching along with a polymerizable resin for priming. The difficulty has been that the compositions are not stable over time (typically no more than 3 months) because the acid component destabilizes the polymerizable resin by hydrolyzing off the functional group to which the backbone of the resin is bonded, rendering the composition useless as a primer after a short time. Such short-term stability is impractical for use in a dental office.

In view of the foregoing, it will be appreciated that it would be a significant improvement over the prior art to provide a two-part self-etching primer composition and a related system that would allow a dental practitioner to mix the composition just prior to application or for short term storage. Such a two-part composition would have a long shelf life, and would allow etching and priming of a prepared dental surface with application of a single composition.

### BRIEF SUMMARY OF THE PREFERRED EMBODIMENTS

The present invention is directed to a two-part self-etching primer composition. The composition includes an acid component comprising a polymerizable organic acid contained in a first part, and a polymerizable component comprising at least one acrylate or methacrylate resin in a second part. A self-etching primer composition is formed upon mixing the first part with the second part.

The polymerizable organic acid of the first part comprises a dental etching agent. Examples of such polymerizable organic acids include, but are not limited to, methacrylic acid, acrylic acid, a diacrylic or polyacrylic acid, and 5-sulfosalicylic acid. One or more mineral acids, such as phosphoric acid, nitric acid, hydrochloric acid, or sulfuric acid may optionally also be included in the first part.

The polymerizable organic acid included in the first part preferably comprises between about 10% and about 50% by weight of the first part, more preferably between about 16% and about 35%, and most preferably between about 20% and about 30% by weight of the first part. In one formulation, the remainder of the first part may comprise water. One or more solvents (e.g., ethanol or acetone) may also be included in the first part.

The polymerizable component of the second part comprises at least one methacrylate or acrylate resin. In one example, one or more included resins may be phosphated, which promotes stronger bonding between the tooth, the self-etching primer, and an adhesive.

The second part of the two-part composition may also include a solvent (e.g., ethanol or acetone), a flavorant or scent (e.g., almond oil) that does not interfere with bonding, and one or more photoinitiators (e.g., Irgacure 819 or camphor quinone). Compositions that include camphor quinone as a photoinitiator may also include a tertiary amine co-initiator.

In a related inventive system and method, the two-part composition is contained within a mixing system. The acidic first part of the composition as described above is contained within a first compartment of the system, while the second part is initially contained within a second compartment of the system. The first and second compartments may be separated from each other (thus separating the first and second parts of the composition) by a valve or rupturable barrier. When it is desired to mix the first part with the second part and form a self-etching primer composition, the contents of the first compartment are mixed with the contents of the second compartment.

For example, in one example the syringe system may comprise a syringe-to-syringe mixing system including a first syringe (having a first compartment) and a second syringe (having a second compartment). The distal delivery ends of the first and second syringes are configured to be coupleable together so as to allow a user to press the plunger of one syringe, expelling the syringe contents into the other syringe, where the first and second parts begin to mix together. The user may press the plunger of the other syringe, cycling the first and second parts back into the first syringe. The first and second parts may be cycled back and forth as many times as needed to mix the components together. After mixing, the mixed self-etching primer composition may be forced into one of the syringes, and the syringes may be separated. The self-etching primer composition within the syringe may be stored short term, and/or may be immediately applied to a tooth surface to be etched and primed. For a light curable self-etching primer, once applied, the composition may be exposed to light curing wavelengths so as to at least partially polymerize the polymerizable organic acid and the acrylate or methacrylate resin within the self-etching primer composition. The system and method allows etching and priming with a single composition that can be easily mixed and then applied to a desired tooth surface.

Syringe-to-syringe mixing systems are one example of a suitable mixing system. Alternative systems include, but are not limited to, a syringe-in-syringe mixing system or a foil pouch mixing system. Examples of syringe-in-syringe mixing systems are disclosed in U.S. Pat. Nos. 2,869,543; 3,326,215; 3,548,825; 3,685,514; 4,464,174; 4,693,706; and 5,489,267, which are hereby incorporated by reference with respect to their disclosure of syringe-in-syringe mixing systems. A suitable foil pouch mixing system includes first and second compartments initially separated from each other. Any of the above systems may be used to initially contain the first part of the composition within the first compartment and the second part of the two-part composition within the second compartment. The contents of the compartments may be mixed to form a self-etching primer composition.

The first and second parts may be provided in a desired mixing ratio. In one example, the mixing ratio of the first part to the second part is between about 1:2 and about 1:1 by weight. In one particularly preferred system, the first part and the second Oz part are of substantially equal weight such that the mixing ratio of the first part to the second part is about 1:1.

These and other advantages and features of the present invention will become more fully apparent from the following description and appended claims, or may be learned by the practice of the invention as set forth hereinafter.

### BRIEF DESCRIPTION OF THE DRAWINGS

In order that the manner in which the above recited and other benefits, advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 illustrates an exemplary two-part self-etching primer system including a syringe-to-syringe mixing system;

FIG. 2 illustrates a syringe of the syringe-to-syringe system of FIG. 1 after mixing of the two-part self-etching primer where the mixed self-etching primer is contained within the syringe;

FIG. 3 illustrates a mixed self-etching primer composition being applied to the surface of a person's tooth; and

FIG. 4 illustrates an applied adhesive and the self-etching primer composition being cured with a dental curing light.

### DETAILED DESCRIPTION OF THE PREFERRED EMBODIMENTS

### I. Introduction

As summarized above, the invention is generally related to two-part self-etching primer compositions that include at least one polymerizable organic acid contained in a first part, and a polymerizable component comprising at least one acrylate or methacrylate resin contained in a second part. A self-etching primer composition is formed by mixing the first part with the second part.

### II. Exemplary Two-Part Self-Etching Primer Compositions

A. First Part

The polymerizable organic acid of the first part comprises a dental etching agent. Examples of such polymerizable organic acids include, but are not limited to, methacrylic acid, acrylic acid, a diacrylic or polyacrylic acid, and 5-sulfosalicylic acid. One or more mineral acids, such as phosphoric acid, nitric acid, hydrochloric acid, or sulfuric acid may optionally also be included in the acidic first part.

The polymerizable organic acid is preferably included in an amount in a range of about 10% to about 50% by weight of the first part, more preferably in a range of about 16% to about 35%, and most preferably in a range of about 20% to about 30% by weight of the first part.

The acidic first part of the two-part composition according to the invention typically includes an aqueous carrier. The water may be deionized if desired prior to use in the first part of the two-part composition. In general, water is preferably included in an amount in a range from about 50% to about 90% by weight of the acidic composition, more preferably in a range of about 65% to about 85%, and most preferably in a range of about 70% to about 80% by weight. Additional solvents (e.g., ethanol or acetone) may optionally be included. In one particularly preferred example, the acidic first part of the composition consists essentially of the polymerizable organic acid and water.

The water, along with the phosphated monomer, is believed to play a part in enhancing the etching activity of the acid and enhancing the bonding of the primer to the tooth surface. It is believed that the water interacts with phosphate groups released from the phosphated monomer, which then interacts with calcium within the tooth structure to enhance the etching activity of the acid. In addition, it is believed that the water promotes stronger bonding between the hydrophilic monomer and the primed tooth surface. Although the exact chemistry is not fully understood, it has been found that self-etching primer compositions that include a substantial portion of water (i.e., at least 50%) in the first part and a phosphated acrylate or methacrylate resin in the second part exhibit enhanced etching activity and stronger bonding than compositions including little or no water and/or only non-phosphated resins. Although compositions including a substantial portion of water and at least one phosphated acrylate or methacrylate resin are preferred, compositions including little or no water and/or only non-phosphated resins can be effective and useful, although less preferred.

B. Second Part

The polymerizable component of the second part comprises at least one methacrylate or acrylate resin. At least one of the included resins may be phosphated. It has been found that use of at least one phosphated methacrylate or acrylate resin results in better bond strength between the tooth surface, the self-etching primer, and an adhesive. Examples of polymerizable components include, but are not limited to, hydroxyl ethyl methacrylate, glycerol dimethacrylate, polyethylene glycol diacrylate, and their phosphated derivatives.

The one or more methacrylate or acrylate polymerizable components are preferably included in an amount in a range of about 35% to about 75% by weight of the second part, more preferably in a range of about 40% to about 70%, and most preferably in a range of about 45% to about 60% by weight of the second part.

The second part of the two-part composition may include a carrier (e.g., ethanol or acetone). In one formulation, the carrier is preferably included in an amount in a range of about 20% to about 70% by weight of the second part, more preferably in a range of about 25% to about 65%, and most preferably in a range of about 35% to about 55% by weight of the second part.

The second part of the two-part composition may also include one or more photoinitiators, one or more electron donor co-initiators, and/or a flavorant or scent. Exemplary photoinitiators include camphor quinone and/or Irgacure 819, available from Ciba Specialty Chemicals located in Basel, Switzerland, although others may be used. In formulations where camphor quinone is included as a photoinitiator, it may be advantageous for the formulation to also include a tertiary amine co-initiator (e.g., ethyl (4-dimethylamino) benzoate (EDMAB)). In examples of the two-part composition that do not initially include a photoinitiator, the two-part composition may still be light curable. In one such example, after the self-etching primer is applied, an adhesive is applied. In instances where the after applied adhesive includes one or more photoinitiators, the self-etching primer composition can be initiated by the photoinitiator containing adhesive, and as such, effectively becomes light curable after being applied.

The inclusion of a flavorant may provide a pleasant flavor and scent to the composition, which may otherwise have an odor offensive to some. One particular flavorant that has been found to not interfere with bonding and is able to provide a pleasant flavor and scent is almond oil, which is somewhat surprising as it is hydrophobic. Other flavorants may include wintergreen oil, mint, or fruit flavors.

### III. Exemplary Methods and Systems

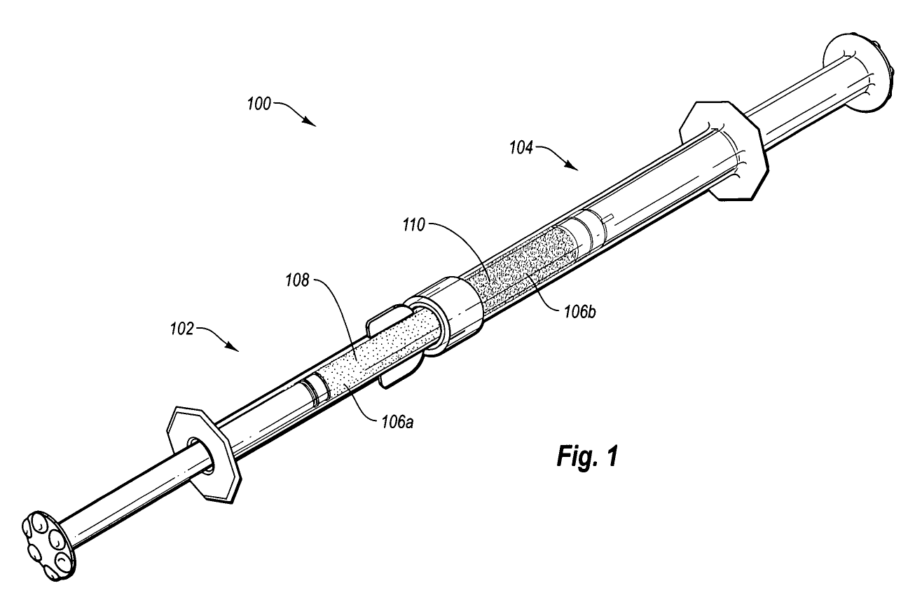

FIG. 1 illustrates an exemplary two-part self-etching primer system 100 including a first syringe 102, a second syringe 104, a composition first part 106a, and a composition second part 106b. The barrel of first syringe 102 defines a first compartment 108, and the barrel of second syringe 104 defines a second compartment 110. First part 106a, which is contained within first compartment 108, comprises an acid component including at least one polymerizable organic acid. Second part 106b is contained within second compartment 110 and comprises a polymerizable component including at least one of an acrylate or methacrylate resin.

The first and second parts may be provided in a desired mixing ratio. In one example, the mixing ratio of the first part to the second part is between about 1:2 and about 1:1 by weight (e.g., 1:1, 2:3, or 1:2). In one particularly preferred system, the first part and the second part are of substantially equal weight such that the mixing ratio of the first part to the second part is about 1:1.

The first and second compartments 108 and 110 may be separated from each other (thus separating the first and second parts of the composition) by a valve or rupturable barrier (not shown). When it is desired to mix the first part with the second part and form a self-etching primer composition, first part 106a within first compartment 108 is mixed with second part 106b within second compartment 110.

In the illustrated system, the distal delivery ends of first syringe 102 and second syringe 104 are coupled together so as to allow a user to press the plunger of one syringe, expelling the syringe contents into the other syringe, where the first and second parts 106a and 106b begin to mix together. The user may press the plunger of the other syringe, cycling the first and second parts back into the first syringe. The first and second parts may be cycled back and forth as many times as needed to mix the components together. Additional syringe mixing systems that may be used in mixing together the two parts are disclosed in U.S. Pat. No. 6,610,034; U.S. application Ser. No. 11/235,461 filed Sep. 26, 2005 and entitled SYRINGE LOCKING STRUCTURES; and U.S. application Ser. No. 10/724,922 filed Dec. 1, 2003 and entitled SYRINGE MIXER AND SYRINGE APPARATUS INCORPORATING THE MIXER. The foregoing U.S. patent and U.S. patent applications are incorporated by reference with respect to their disclosure of syringe mixing systems.

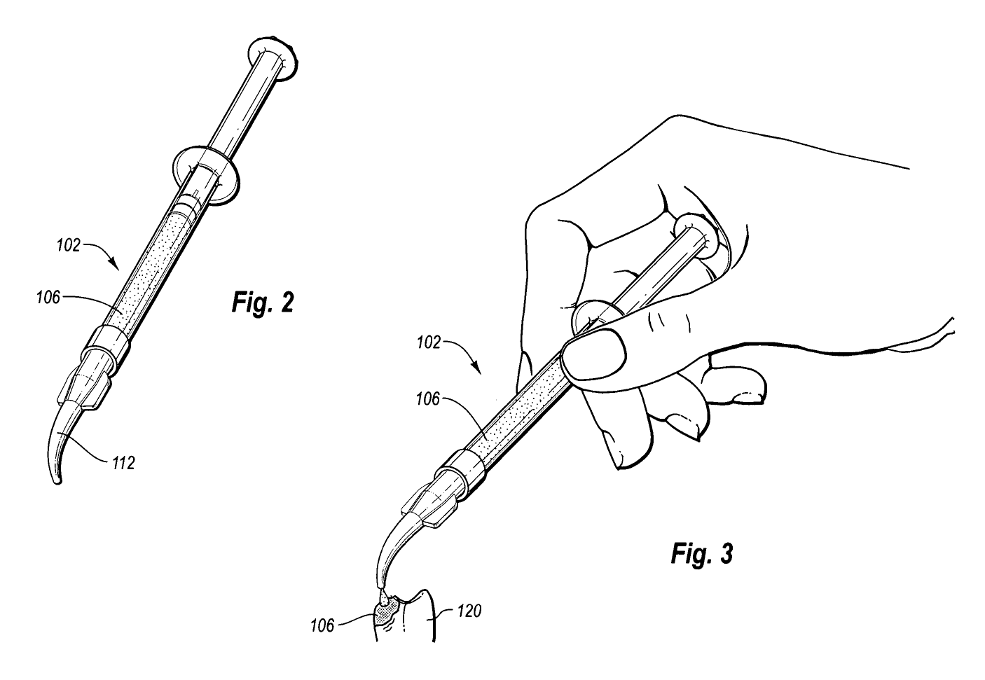

As seen in FIG. 2, once mixed, the mixed self-etching primer composition 106 may be contained within syringe 102. The composition 106 could alternatively be contained within syringe 104. A dispensing tip 112 has been coupled to the distal delivery end of syringe 102.

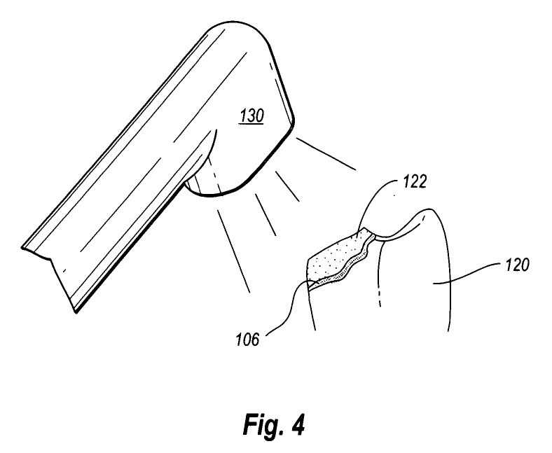

As shown in FIG. 3, the mixed composition 106 may be applied to a tooth surface 120 to be etched and primed. An advantage of the self-etching primer composition 106 is that rinsing of the surface 120 after etching is not required. An adhesive 122 may be applied directly over self-etching primer 106. In order to cure both a light curable adhesive 122 and a light curable self-etching primer composition 106, the adhesive 122 and composition 106 may be exposed to light curing wavelengths generated by a dental curing device 130 (FIG. 4). Adhesive 122 and composition 106 may be cured simultaneously where the layer of self-etching composition 106 is relatively thin. For a thicker layer of a light curable self-etching composition 106, the adhesive 122 and self-etching composition 106 may be cured separately. Exposure to light curing wavelengths in the presence of a photoinitiator at least partially polymerizes the polymerizable organic acid and the acrylate or methacrylate resin within the self-etching primer composition 106, resulting in a strong bond between the tooth tissue, the self-etching primer, and the adhesive 122. The system and method allows etching and priming with a single composition that can be easily mixed and then applied to desired tooth tissue. No rinsing of a dental etching solution is required, and the self-etching primer results in a primed bonding surface on the tooth to which the adhesive can strongly adhere.

Syringe-to-syringe self-etching primer mixing system 100 is one example of a suitable mixing system. Alternative systems include, but are not limited to, a syringe-in-syringe mixing system or a foil pouch mixing system. A syringe-in-syringe mixing system may include first and second compartments for containing the two parts of the two-part self-etching primer composition. The compartments may initially be separated from each other (e.g., by a valve or a rupturable barrier). Examples of syringe-in-syringe mixing systems are disclosed in U.S. Pat. Nos. 2,869,543; 3,326,215; 3,548,825; 3,685,514; 4,464,174; 4,693,706; and 5,489,267, which are hereby incorporated by reference with respect to their disclosure of syringe-in-syringe mixing systems.

A suitable foil pouch mixing system may include first and second compartments initially separated from each other. Each compartment of the foil pouch mixing system may initially contain one part of the two-part composition. The user may simply press the foil pouch so as to cause mixing of the two parts (e.g., through rupture of a thin membrane initially separating the two compartments). Mixing of the two parts results in formation of a self-etching primer composition ready to be applied to a desired tooth surface. Additional mixing systems will be apparent to one skilled in the art.

### IV. EXAMPLES

The following examples set forth various exemplary two-part self-etching primer compositions according to the invention. These examples are intended to be purely exemplary and should not be viewed as limiting the scope of the present invention. Examples that were actually made are set forth in past tense, while hypothetical examples are set forth in present tense.

### Example 1

A two-part self-etching primer composition according to the invention was made by mixing together the following components to form a first part containing an acid component:

| | |
|---|---|
| Water | 75% |
| Methacrylic Acid | 25% |

A second part containing a polymerizable component was formed by mixing together the following components:

| | |
|---|---|
| Phosphated HEMA | 24% |
| Phosphated GDMA | 8.2% |
| Ethanol | 48% |
| Camphor Quinone | 0.4% |
| EDMAB | 0.8% |
| Irgacure 819 | 0.7% |
| Almond Oil | 0.3% |
| PEGDA | 17.6% |

The first and second parts were mixed together in a 1:1 mixing ratio to form a self-etching primer composition. The two-part self-etching primer was stable, having a shelf life of 18 months or more. The two-part composition was mixed to form a self etching primer. The self-etching primer was effective in etching and priming a tooth surface preparatory to applying an adhesive. No rinsing of the self-etching primer was needed prior to application of the adhesive. In use, the self-etching primer promoted strong bonding between the tooth surface and the adhesive.

### Example 2

A two-part self-etching primer composition according to the invention was made by mixing together the following components to form a first part containing an acid component:

| | |
|---|---|
| Water | 75% |
| Methacrylic Acid | 25% |

A second part containing a polymerizable component was formed by mixing together the following components:

| | |
|---|---|
| Phosphated HEMA | 24% |
| Phosphated GDMA | 8.2% |
| Ethanol | 48% |
| Camphor Quinone | 0.4% |
| EDMAB | 0.8% |
| Irgacure 819 | 0.7% |
| Almond Oil | 0.3% |
| PEGDA | 17.6% |

The first and second parts were mixed together in a 2:3 mixing ratio to form a self-etching primer composition. The two-part self-etching primer was stable, having a shelf life of 18 months or more. The two-part composition was mixed to form a self etching primer. The self-etching primer was effective in etching and priming a tooth surface preparatory to applying an adhesive. No rinsing of the self-etching primer was needed prior to application of the adhesive. In use, the self-etching primer promoted strong bonding between the tooth surface and the adhesive.

### Example 3

A two-part self-etching primer composition according to the invention was made by mixing together the following components to form a first part containing an acid component:

| | |
|---|---|
| Water | 75% |
| Methacrylic Acid | 25% |

A second part containing a polymerizable component was formed by mixing together the following components:

| | |
|---|---|
| Phosphated HEMA | 24% |
| Phosphated GDMA | 8.2% |
| Ethanol | 48% |
| Camphor Quinone | 0.4% |
| EDMAB | 0.8% |
| Irgacure 819 | 0.7% |
| Almond Oil | 0.3% |
| PEGDA | 17.6% |

The first and second parts were mixed together in a 1:2 mixing ratio to form a self-etching primer composition. The two-part self-etching primer was stable, having a shelf life of 18 months or more. The two-part composition was mixed to form a self etching primer. The self-etching primer was effective in etching and priming a tooth surface preparatory to applying an adhesive. No rinsing of the self-etching primer was needed prior to application of the adhesive. In use, the self-etching primer promoted strong bonding between the tooth surface and the adhesive.

### Example 4

A two-part self-etching primer composition according to the invention was made by mixing together the following components to form a first part containing an acid component:

| | |
|---|---|
| Water | 75% |
| Methacrylic Acid | 25% |

A second part containing a polymerizable component was formed by mixing together the following components:

| | |
|---|---|
| Phosphated HEMA | 24% |
| Phosphated GDMA | 8.2% |
| Ethanol | 49.9% |
| Almond Oil | 0.3% |
| PEGDA | 17.6% |

The first and second parts were mixed together in a 1:1 mixing ratio to form a self-etching primer composition. The two-part self-etching primer was stable, having a shelf life of 18 months or more. The two-part composition was mixed to form a self etching primer. The self-etching primer was effective in etching and priming a tooth surface preparatory to applying an adhesive. No rinsing of the self-etching primer was needed prior to application of the adhesive. In use, the self-etching primer promoted strong bonding between the tooth surface and the adhesive.

### Example 5

A two-part self-etching primer composition according to the invention was made by mixing together the following components to form a first part containing an acid component:

| | |
|---|---|
| Water | 75% |
| Methacrylic Acid | 25% |

A second part containing a polymerizable component was formed by mixing together the following components:

| | |
|---|---|
| Phosphated HEMA | 24% |
| Phosphated GDMA | 8.2% |
| Ethanol | 48.7% |
| Camphor Quinone | 0.4% |
| EDMAB | 0.8% |
| Almond Oil | 0.3% |
| PEGDA | 17.6% |

The first and second parts were mixed together in a 1:1 mixing ratio to form a self-etching primer composition. The two-part self-etching primer was stable, having a shelf life of 18 months or more. The two-part composition was mixed to form a self etching primer. The self-etching primer was effective in etching and priming a tooth surface preparatory to applying an adhesive. No rinsing of the self-etching primer was needed prior to application of the adhesive. In use, the self-etching primer promoted strong bonding between the tooth surface and the adhesive.

### Comparative Example 6

A one-part self-etching primer composition was made by mixing together the following components:

| | |
|---|---|
| Water | 32.12% |
| Methacrylic Acid | 8.57% |
| Phosphated HEMA | 10.71% |
| Phosphated GDMA | 5.35% |
| Ethanol | 32.12% |
| Almond Oil | 0.32% |
| PEGDA | 10.71% |
| BHT | 0.11% |

BHT was included as a stabilizer in an attempt to form a stable one part composition. This formula proved to be unstable after about 6 weeks storage in a 40° C. oven.

### Example 7

A two-part self-etching primer composition according to the invention is made mixing together the following components to form a first part containing an acid, component:

| | |
|---|---|
| Water | 65% |
| Methacrylic Acid | 35% |

A second part containing a polymerizable component is formed by mixing together the following components:

| | |
|---|---|
| Phosphated HEMA | 15% |
| Phosphated GDMA | 15% |
| Ethanol | 55% |
| PEGDA | 15% |

The first and second parts are mixed together in a 2:3 mixing ratio to form a self-etching primer composition. The two-part self-etching primer is stable, having a shelf life of 18 months or more. The two-part composition is mixed to form a self etching primer. The self-etching primer is effective in etching and priming a tooth surface preparatory to applying an adhesive. No rinsing of the self-etching primer is needed prior to application of the adhesive. In use, the self-etching primer promotes strong bonding between the tooth surface and the adhesive.

### Example 8

A two-part self-etching primer composition according to the invention is made by mixing together the following components to form a first part containing an acid component:

| | |
|---|---|
| Water | 84% |
| Methacrylic Acid | 16% |

A second part containing a polymerizable component is formed by mixing together the following components:

| | |
|---|---|
| Phosphated HEMA | 35% |
| Phosphated GDMA | 15% |
| Ethanol | 32.6% |
| PEGDA | 15% |
| Camphor Quinone | 0.4% |
| EDMAB | 0.8% |
| Irgacure 819 | 0.7% |
| Almond Oil | 0.5% |

The first and second parts are mixed together in a 1:2 mixing ratio to form a self-etching primer composition. The two-part self-etching primer is stable, having a shelf life of 18 months or more. The two-part composition is mixed to form a self etching primer. The self-etching primer is effective in etching and priming a tooth surface preparatory to applying an adhesive. No rinsing of the self-etching primer is needed prior to application of the adhesive. In use, the self-etching primer promotes strong bonding between the tooth surface and the adhesive.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A two-part self-etching primer composition, comprising:
an acid component contained in a first part, the acid component including at least one polymerizable organic acid; and
a polymerizable component comprising at least one of an acrylate or methacrylate resin contained in a second part;
wherein a self-etching primer composition is formed by mixing the first part and second part together.

2. A two-part self-etching primer composition as recited in claim 1, the polymerizable organic acid comprising at least one organic acid selected from the group consisting of methacrylic acid, acrylic acid, a diacrylic acid, a polyacrylic acid, and 5-sulfosalicylic acid.

3. A two-part self-etching primer composition as recited in claim 1, the polymerizable organic acid having a concentration in a range of about 10% to about 50% by weight of the first part.

4. A two-part self-etching primer composition as recited in claim 1, the polymerizable organic acid having a concentration in a range of about 16% to about 35% by weight of the first part.

5. A two-part self-etching primer composition as recited in claim 1, the polymerizable organic acid having a concentration in a range of about 20% to about 30% by weight of the first part.

6. A two-part self-etching primer composition as recited in claim 1, wherein the first part further comprises water.

7. A two-part self-etching primer composition as recited in claim 6, the water having a concentration in a range of about 50% to about 90% by weight of the first part.

8. A two-part self-etching primer composition as recited in claim 6, the water having a concentration in a range of about 65% to about 85% by weight of the first part.

9. A two-part self-etching primer composition as recited in claim 6, the water having a concentration in a range of about 70% to about 80% by weight of the first part.

10. A two-part self-etching primer composition as recited in claim 1, wherein the first part consists essentially of at least one polymerizable organic acid and water.

11. A two-part self-etching primer composition as recited in claim 1, the polymerizable component of the second part comprising at least one phosphated acrylate or phosphated methacrylate resin.

12. A two-part self-etching primer composition as recited in claim 1, the second part further comprising at least one photoinitiator.

13. A two-part self-etching primer composition as recited in claim 1, the second part further comprising at least one solvent.

14. A two-part self-etching primer composition as recited in claim 13, the at least one solvent comprising ethanol.

15. A two-part self-etching primer composition as recited in claim 1, the second part further comprising at least one flavorant.

16. A two-part self-etching primer composition as recited in claim 1, wherein two-part self-etching primer composition is light curable.

17. A two-part self-etching primer system, comprising:
a mixing system including a first compartment and a second compartment;
an acid component contained within the first compartment, the acid component including at least one polymerizable organic acid; and
a polymerizable component contained within the second compartment, the polymerizable component including at least one of an acrylate or methacrylate resin;
wherein a self-etching primer composition is formed by mixing the contents of the first compartment with the contents of the second compartment.

18. A system as recited in claim 17, wherein the mixing system comprises a syringe-to-syringe mixing system including a first syringe having a first compartment for initially containing the acid component and a second syringe having a second compartment for initially containing the polymerizable component.

19. A system as recited in claim 17, wherein the mixing system comprises a syringe-in-syringe mixing system including a first compartment for initially containing the acid component and a second compartment for initially containing the polymerizable component.

20. A system as recited in claim 17, wherein the mixing system comprises a foil pouch mixing system including a first compartment for initially containing the acid component and a second compartment for initially containing the polymerizable component.

21. A system as recited in claim 17, further comprising a valve or rupturable membrane separating the first compartment from the second compartment.

22. A system as recited in claim 17, the first part having a first weight and the second part having a second weight such that the mixing ratio of the first part to the second part is between about 1:2 and about 1:1 by weight.

23. A system as recited in claim 17, the first part and the second part having substantially equal weights such that the mixing ratio of the first part to the second part is about 1:1 by weight.

24. A method of etching and priming a tooth using a two-part self-etching primer composition, comprising:
providing a two-part self-etching primer composition comprising:
an acid component included in a first part, the acid component including at least one polymerizable organic acid; and
a polymerizable component comprising at least one of an acrylate or methacrylate resin included in a second part;
mixing the first part and second part of the composition together so as to form a self-etching primer composition;
applying the mixed composition to a tooth surface to be etched and primed; and
exposing the mixed composition on the tooth surface to one or more light curing wavelengths so as to at least partially polymerize the polymerizable organic acid and the acrylate or methacrylate resin within the mixed composition.

25. A method as recited in claim 24, further comprising applying an adhesive to the etched and primed tooth surface prior to exposing the mixed composition on the tooth surface to the one or more light curing wavelengths.
